(19) United States
(12) Patent Application Publication
(10) Pub. No.: US 2003/0017971 A1
(43) Pub. Date: Jan. 23, 2003

(54) Method for increasing levels of export of mRNA from the nucleus to the cytoplasm by providing in the nucleus recombinant mammalian export protein ALY

(75) Inventors: Ming-Juan Luo, Roslindale, MA (US); Zhaolan Zhou, Malden, MA (US); Robin Reed, Belmont, MA (US)

Correspondence Address: Dike, Bronstein, Roberts & Cushman, Intellectual Property Practice Group, EDWARDS & ANGELL, LLP, P.O. Box 9169, Boston, MA, 02209, US

(73) Assignee: The President and Fellows of Harvard College

(21) Appl. No.: 09/945,248

(22) Filed: Aug. 31, 2001

Related U.S. Application Data
(60) Provisional application No. 60/229,765, filed on Sep. 1, 2000.

(51) Int. Cl. A61K 48/00; C12N 5/06

(52) U.S. Cl. 514/8; 514/44; 435/325

(57) ABSTRACT

The present invention features methods for increasing levels of nuclear export of mRNA by providing recombinant mammalian export factor. Also featured are methods for preparing metazoan or mammalian cells with recombinant factor ALY that are useful for facilitating mRNA export. The export factor ALY binds mRNA in a non sequence specific manner and the ALY-mRNA complex is exported more efficiently than unbound mRNA. The addition of excess ALY to metazoan or mammalian cell results in increased mRNA export, increased gene expression and increased production of recombinant proteins.

The present application claims the benefit of U.S. provisional application No. 60/229,765, filed Sep. 1, 2000, which is incorporated herein by reference in its entirety.

### FIELD OF THE INVENTION

The present invention relates to methods for obtaining higher levels of gene expression from cDNAs, particularly to increasing export of mRNA from the nucleus of a mammalian cell. More particularly, it relates to mammalian cells containing recombinant cDNA and recombinant mammalian export factor ALY that facilitates export of the mRNA derived from the cDNA. Increased export rate and efficiency can be obtained for mRNA with an intron or for mRNA lacking an intron in the presence of recombinant cellular factor ALY.

### BACKGROUND OF THE INVENTION

The biopharmaceutical industry has been built on the success of developing protein agents as therapeutics, e.g. for treating diseases in humans and animals. The development of biopharmaceuticals has been driven by the use of recombinant DNA technology or genetic engineering to clone and express the proteins of interest and engineer their manufacture at commercial scale. Virtually all recombinant proteins have been expressed from cDNA in which the introns have been removed by splicing. There are several critical steps that limit gene expression including the export of mRNA from the nucleus into the cytoplasm. Development of improved methods to export mRNA transcribed from cDNA from the nucleus is an ongoing process and will continue to increase the amount of recombinant protein obtained.

Work over the last 20 years has indicated that the presence of an intron(s) in a gene is required for maximal levels of gene expression (Luo and Reed, PNAS (1999) 96:14937-42 and references therein). Because most introns are extremely large (10⁴-10⁵ base pairs), typically, it is not technically practical to use natural genes containing introns for gene expression. Several methods have been developed to circumvent the requirement for an intron. For example, some commercially available vectors for gene expression contain a small intron. Recently, vectors containing the CMV promoter allow gene expression in the absence of an intron. However, in the presence of an intron, gene expression increases even when a CMV promoter is used.

The role of the intron in promoting maximal gene expression appears to be general. Specifically, mRNA generated by splicing is more efficiently exported than the corresponding mRNA already lacking the intron. The process of splicing (excising the intron from the gene sequence) promotes efficient mRNA transport out of the nucleus. Luo and Reed (PNAS (1999) 96:14937-42) reported that mRNA generated by splicing is more efficiently exported than the corresponding mRNA lacking the intron. This result indicated that splicing and mRNA export are coupled.

It has been reported that the essential yeast factor Yra1p specifically binds to the essential yeast export factor Mex67p. Mex67p and Mtr2p constitute an essential mRNA export complex that interacts with poly(A)+ RNA and nuclear pore proteins (Sträβer and Hurt, EMBO J (2000) 19:410). Suppression of Yra1p induces a decrease in mRNA export whereby Yra1p is implicated as an export factor in the Mex67p export pathway. However the mode of Yra1p activity in the RNA export pathway was not determined.

A nuclear protein, termed ALY, was identified and characterized by Bruhn et al. (GENES DEV (1997) 11:640-53). The mammalian factor ALY has been produced by transcription and translation in vitro of cDNA cloned from an embryonic day 10 mouse (Bruhn, GENES DEV (1997) 11:640-53). It was reported that ALY interacts specifically with two TCRα enhancer-binding factors, LEF-1 and AML-1.

Stutz et al. report that REF, an evolutionary conserved family of hnRNP-like proteins, interacts with TAP/Mex67p and postulates that REF participates in MRNA nuclear export (Stutz, RNA (2000) 6:638-50). However, Stutz does not provide any experimental evidence or suggest that such experimental evidence exists which supports the unsubstantiated postulate. Murine REF proteins exhibited binding to a RNA probe in vitro which was incubated with purified recombinant REF and assayed by electrophoretic gel mobility retardation. The REF family appear to exhibit a general affinity for RNA and single-stranded DNA. There are no reported attempts to isolate or substantially purify a complex comprising REF and RNA.

Recently, much attention has been focused on developing methodologies for maximizing gene expression (particularly in mammalian cells) from cDNA vectors that lack an intron. Export factors for RNA in yeast have been identified and their mode of function in the RNA export pathway has been tentatively identified. Identification of related export factors in metazoan systems has been more difficult and at present no factors that specifically promote mRNA export from the nucleus to the cytoplasm of metazoan have been identified. The discovery of factors and methods that specifically and substantially increase the rate of mRNA export from the nucleus of a metazoan cell has not been obtained. The ability to obtain high levels of recombinant proteins and increased gene expression from cDNA in mammalian cells is, in part, limited by slow mRNA export processes. Thus, there exists a great need for new methods to increase export of recombinant mRNA from the cell nucleus to the cytoplasm.

### SUMMARY OF THE INVENTION

The present invention provides metazoan cells, particularly mammalian cells, containing recombinant cDNA and recombinant cellular factor ALY, which promotes mRNA export, and provides methods for the increased export of recombinant mRNA from the nucleus of such cells. The export process is promoted by providing recombinant cellular factor ALY in the metazoan cell whereby exporting of mRNA from the nucleus of the cell is increased over compared export from a similar cell without recombinant cellular factor ALY. The methods of the present invention can be used to increase the levels of recombinant gene expression and to obtain increased quantities of recombinant protein wherein the increased export of mRNA alleviates one of the limiting factors for gene expression in these processes.

In one aspect of the invention, the mRNA that is to be exported from the nucleus of the cell forms a stable binding complex containing both ALY and mRNA wherein the binding of ALY is non-specific to particular mRNA sequences. In one embodiment, a therapeutic agent comprising the mRNA-ALY binding complex facilitates mRNA export thereby alleviating disorders stemming from insufficient mRNA export. Alternatively, the complex can be used to increase gene expression or to obtain higher levels of recombinant protein that is isolated and used in subsequent applications.

In accord with the invention, the cellular factor ALY binds to mRNA by at least two pathways. One pathway involves the splicing of the intron from the pre-mRNA sequence wherein binding can be mediated by the splicing-machinery. In an alternative pathway, cellular factor ALY binds directly to mRNA without any involvement of an intron.

In a preferred embodiment, a metazoan cell or mammalian cell comprises excess recombinant cellular factor ALY wherein the excess recombinant cellular factor ALY facilitates export of mRNA from the nucleus to the cytoplasm of the cell. The recombinant cellular factor ALY localizes in the nucleus of the cell. The addition of excess recombinant cellular factor ALY to a cell increases the export of mRNA in a dose-dependant manner

In preferred embodiments of the invention, the recombinant cellular factor ALY is introduced into the metazoan or mammalian cell by microinjection of ALY into the cytoplasm or nucleus of the cell. Alternatively, a vector that expresses cellular factor ALY is introduced into the cytoplasm or nucleus of the metazoan or mammalian cell. Methods of introducing the vector are not particularly limited. Preferably, the vector is introduced by microinjection. Typically, at least a portion of the ALY injected into the cytoplasm or expressed from a vector in the cytoplasm diffuses into the nucleus.

In other preferred embodiments, the metazoan or mammalian cell line is fused with a cell line stably expressing the recombinant cellular factor ALY wherein the recombinant cellular factor ALY is imported into the metazoan or mammalian cell nuclei However, other methods for the introduction of recombinant cellular factor ALY into the cell also are acceptable.

In one preferred embodiment of the invention, the cellular factor ALY is the murine homolog of the essential yeast mRNA export factor Yra1p. However, homologs of the essential yeast mRNA export factor Yra1p derived from other mammalian sources, including recombinant human ALY, also can be used to facilitate the export of recombinant mRNA from the cell nucleus. Recombinant human cellular factor ALY is a preferred homolog.

The present invention provides a method for facilitating the export of mRNA from the nucleus of a cell to the cytoplasm comprising the introduction of recombinant cellular factor ALY into a cell containing cDNA, wherein the recombinant cellular factor ALY localizes in the nucleus thereby associating with mRNA transcribed from the cDNA to form a mRNA-ALY binding complex, thereby facilitating the export of said mRNA from the nucleus.

In another preferred embodiment of the invention, pre-mRNAs contain introns that are removed by splicing. Recombinant ALY binds to mRNP complexes generated by splicing. Preferably, the ALY complexes with the mRNA during spliceosome assembly and becomes tightly associated with the spliced mRNP. Thus, the recombinant ALY can increase both the rate and efficiency of mRNA transport or export from the nucleus to the cytoplasm of the cell. The quantity of recombinant cellular factor ALY introduced into the metazoan cell for export of spliced mRNA typically is greater than 5 ng, preferably greater than 25 ng, or more preferably greater than 75 ng.

We have discovered that recombinant ALY can also be used to increase the export of mRNA from pre-mRNAs lacking an intron. In this case an excess of the recombinant cellular factor ALY over that required for exporting mRNA formed from pre-mRNAs containing an intron is required to obtain the same affect. The quantity of excess recombinant cellular factor ALY introduced into the metazoan cell for export of mRNA that does not contain an intron preferably is greater than 75 ng.

The present invention further provides a method for increasing the level of gene expression wherein the recombinant cellular factor ALY is introduced into a metazoan or mammalian cell, localizes in the nucleus such that the recombinant cellular factor ALY associates with the transcribed mRNA sequence forming an mRNA-ALY binding complex and facilitating export of said mRNA sequence out of the nucleus thereby increasing the level of gene expression. The increased export of mRNA can cause higher levels of protein production that is the equivalent of increasing the level of gene expression in a similar system without ALY. Non-limiting examples of possible sources for mRNA transcription can include innate DNA, exogenous cDNA, exogenous plasmids, and the like.

The invention additionally provides a method for obtaining increased levels of recombinant proteins wherein the recombinant cellular factor ALY is introduced into a metazoan or mammalian cell, localizes in the nucleus wherein the recombinant cellular factor ALY associates with the transcribed mRNA sequence forming an mRNA-ALY binding complex and facilitates export of said mRNA sequence out of the nucleus. The increased level of exported mRNA increases protein transcription thereby permitting the purification of higher levels of recombinant protein.

### BRIEF DESCRIPTION OF THE FIGURES

FIGS. 1A-B shows the binding of cellular factor ALY to mRNA. A) shows the assay of spliced mRNA and nonspliced mRNA during purification by gel filtration and MBP affinity chromatography for injection into Xenopus oocyte nuclei. B) is the export assays of MBP-affinity purified spliced mRNA and nonspliced mRNA mixed with U6 snRNA measuring RNA content of the nucleus and cytoplasm.

FIGS. 2A-B illustrates the splicing dependent recruitment of ALY to mRNA. Western blots of MBP-affinity purified mRNPs or the hnRNP complex H, each containing 10 ng of spliced mRNA, nonspliced mRNA or pre-mRNA, were probed with the indicated antibodies. Nuclear extract was loaded in lane 1. Complexes were assembled on AdML (A) or Ftz RNAs (B).

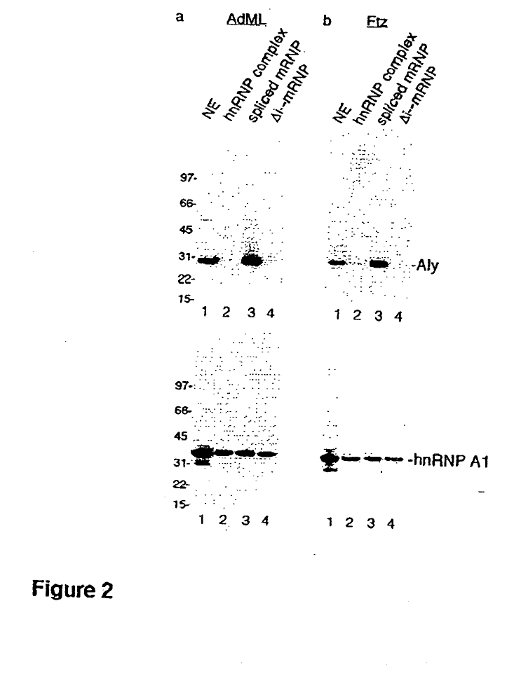

FIGS. 3A-E is the determination of timing, specificity and stability of ALY recruitment. A) analysis of mRNA from the eluted complexes. B-E) Western blots of total protein from the eluted complexes shown in A were probed with the indicated antibodies.

FIGS. 4A-C shows that ALY promotes export of mRNA generated by the spliceosome. A) analysis of a mixture of U6 snRNA and AdML spliced or nonspliced mRNA. B) analysis of RNA in the nucleus and cytoplasm after injecting of U6 snRNA and AdML spliced or nonspliced mRNA mixture into Xenopus oocyte nuclei containing either GST-ALY (25 or 75 ng) or buffer alone (control) and incubating the oocytes for 45, 90, and 135 minutes. C) nuclei from 90 minute incubation with either buffer or 25 ng GST-ALY were isolated, homogenized in binding buffer, rotated with glutathione beads overnight and the total RNA in bound and unbound fractions was analyzed.

FIGS. 5A-B illustrates that ALY is present in nuclear speckle domains and shuttles between the nucleus and cytoplasm. A) Heterokaryon assays were preformed by co-culturing Xenopus and murine cell lines and hnRNP A1, hnRNP C and ALY were detected with arrows indicating Xenopus nuclei. B) affinity purified ALY or SC35 antibodies were used for double indirect immunofluorescence of HeLa cells. Individual and merged cells are shown.

FIG. 6 illustrates the nucleotide sequence of murine ALY cDNA and the predicted amino acid sequence of the encoded protein.

### DETAILED DESCRIPTION OF THE INVENTION

The present invention provides metazoan cells, particularly mammalian cells, containing recombinant factors that promote mRNA export and methods to facilitate increased rate and/or efficiency of export of mRNA from the nucleus of said cells. The export process is promoted by providing an increasing amount of recombinant cellular factor ALY in the metazoan cell. The recombinant cellular factor ALY facilitates the export of mRNA from the nucleus of the cell.

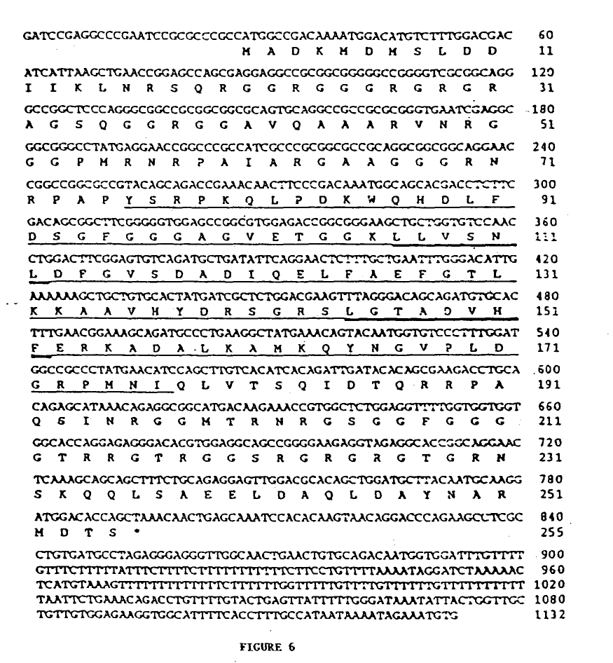

As used herein, the term ALY refers to a protein derived from the DNA sequence set forth in FIG. 6 and to the deduced amino acid sequence illustrated in FIG. 6, and also to homologous proteins found in other metazoan and mammalian cells. The degree of homology required is that of other species to species homology for other genes. This discovery paves the way for the identification of like cellular proteins in all species.

The murine protein ALY that is used by way of example herein can be made according to the procedures described by Bruhn et al (GENES DEV (1997) 11:640-53). Additional vectors and procedures for producing ALY can be found in Stutz et al. (RNA (2000) 6:638-650). As referred to herein, and as is well known in the art, GST-ALY is the fusion protein comprising GST and ALY. As referred to herein, and as is well known in the art, HIS-ALY is the fusion protein comprising a polyhistidine domain and ALY wherein the polyhistidine domain preferably has between 4 and 24 histidine residues or more preferably 6 histidine residues. As referred to herein, the term “metazoan cell” or “metazoan cell line” refer to any cell or cell line that originates in any animal more complex than one-celled protozoa. Preferred metazoan cells are insect, mammalian or amphibian cells. Particularly useful metazoan cells are Xenopus oocyte cells. As referred to herein, and as is well known in the art, Xenopus oocyte cells are egg cells from Xenopus laevis. “Spliced mRNA,” as used herein, refers to mRNA formed from pre-mRNA containing an intron by means of the cell spliceosome assembly mechanism. mRNP containing “spliced mRNA” is significantly different from mRNP formed from pre-mRNA lacking an intron wherein the splicing mechanisms of the cell are not used.

In one aspect of the invention, the facilitation of spliced mRNA export from the nucleus occurs with recruitment of recombinant cellular factor ALY to mRNA wherein the recruitment process is promoted by the splicing machinery to a spliceosome wherein recruitment occurs during splicing of mRNA containing an intron. The splicing-dependent recruitment process generates a stable binding complex, referred to herein as the ALY-mRNA binding complex, comprising both recombinant cellular factor ALY and mRNA wherein ALY exhibits non-sequence specific binding.

The presence of ALY in the mRNP was confirmed by Western analysis using ALY antibodies wherein the antibodies recognized a single band of the correct size (30 kD) corresponding to ALY (Bruhn, GENES DEV (1997) 11:64053; Wichmann Hum Immunol (1999) 60:57-62).

The strength of ALY association to the spliceosome was measured by isolation of spliceosome complexes under low (60 mM) and high (250 mM) salt conditions at several time intervals. In low salt conditions, ALY was first detected in the 7 minute spliceosome and was more abundant at later time points. In high salt conditions, ALY is associated at high levels of spliced mRNA (after 40 minutes). The recombinant cellular factor ALY is recruited during spliceosome assembly and then becomes more tightly associated with the spliced mRNA.

The ALY-mRNA binding complex was isolated by injection of spliced mRNA into Xenopus oocyte nuclei and subsequent isolation and purification of the exported mRNP by gel-filtration and maltose binding protein (MBP) affinity chromatography described in Example 1. Other methods for the preparation and purification of ALY-mRNA binding complexes would also be acceptable including but not limited to in vivo and in vitro methods

The substantially purified ALY-mRNA binding complex is at least 50% pure by weight. Preferably, the substantially purified ALY-mRNA binding complex is at least 80% pure by weight and most preferably the substantially purified ALY-mRNA binding complex is at least 95% pure by weight.

To increase export of mRNA derived from pre-mRNA lacking an intron, i.e., “nonspliced mRNA,” typically requires the addition of excess recombinant cellular factor ALY over that required for the same level of exporting of spliced mRNA. In the presence of excess recombinant cellular factor ALY, efficient ALY binding to mRNA occurs by direct recruitment of ALY to nonspliced mRNA bypassing the splicing-dependent pathway. Contacting nuclear extracts of Xenopus nuclei comprising mRNA and recombinant cellular factor ALY-GST with glutathione beads resulted in mRNA binding to the beads via intermediary ALY-mRNA binding complex (see Example 3).

The terms “nonspliced mRNA” and “nonspliced mRNP”, for the purposes of the specification and claims, are meant to refer to respective mRNA and mRNP that was generated from pre-mRNAs without an intron. The plasmids encoding nonspliced mRNA were constructed by exact deletion of the intron for each pre-mRNA (Luo and Reed, PNAS (1999) 96:14937-42).

The preferred level of recombinant cellular factor ALY present in a metazoan cell to effectively bind mRNA is greater than 5 ng, more preferably greater that 25 ng, and most preferably greater than 75 ng. For nonspliced mRNA, the preferred level of recombinant cellular factor ALY present in a metazoan or mammalian cell to effectively bind mRNA preferably is greater than 75 ng, more preferably greater that 150 ng.

For the purposes of the specification and claims mRNA can be any type of mRNA derived from cellular or viral genes. Preferably, the mRNA is derived from mammalian genes.

An ALY-mRNA binding-complex of the present invention can have applications as a therapeutic agent where such a therapeutic agent alleviates cellular disfunctions stemming from poor mRNA export. Preferably, the therapeutic agent comprises a mammalian cellular factor for use in veterinary application or a human cellular factor for application within the human body. The therapeutic agent typically is provided in a pharmaceutically acceptable carrier and preferably the composition is non-pyrogenic. Alternatively, the complex can be used to increase the level of gene expression or to obtain higher levels of recombinant proteins for subsequent applications.

Preferably, the recombinant cellular factor ALY is introduced into the metazoan cell by microinjection of the ALY into the cytoplasm of the cell with subsequent localization into the nucleus during an incubation period. The amount of recombinant cellular factor ALY introduced into the cytoplasm by microinjection in accord with the present invention typically is about 5 ng or greater. Preferably, the amount of recombinant cellular factor ALY introduced into the cytoplasm by microinjection is about 25 ng or greater. More—preferably, the amount of recombinant cellular factor ALY introduced into the cytoplasm by microinjection is about 75 ng or greater. The incubation period preferably is at least 30 minutes. More preferably, the incubation period is at least 2 hours. Most preferably, the incubation period is at least 8 hours.

Preferably, a vector expressing cellular factor ALY is introduced into the metazoan cell by microinjection into the cytoplasm or nucleus of the metazoan cell. Preferably injection of the vector into the metazoan or mammalian cell results in a stable cell line that stably expresses recombinant cellular factor ALY. Typically at least a portion of the expressed cellular factor ALY shuttles between the nucleus and the cytoplasm such that at least a portion of the ALY prepared in the cytoplasm is transported to the nucleus. In general the amount of recombinant cellular factor ALY expressed from the vector is typically about 5 ng or greater. Preferably, the amount of recombinant cellular factor ALY expressed from the vector is about 25 ng or greater. More —preferably, the amount of recombinant cellular factor ALY expressed from the vector is about 75 ng or greater.

In a preferred embodiment of the invention, recombinant cellular factor ALY is introduced directly into the nucleus of a metazoan cell by fusing the metazoan cell line with a cell line that stably expresses cellular factor ALY. The cells can be fused, for example, in the presence of polyethylene glycol (PEG), or by any procedure known to those skilled in the art. Cellular factor ALY is expressed and subsequently localizes in the nucleus of the metazoan cell.

The cell line stably expressing cellular factor ALY can be any cell line containing an appropriate vector for the expression of either ALY, or a fused protein of ALY such as, for example, ALY-GFP, HIS-ALY or GST-ALY. Preferably, the cell line used to express the ALY is a mammalian cell line. Particularly preferred cell lines that are useful for expressing a fused protein containing ALY are HeLa cells and murine fibrosarcoma cell line L929 which stably expresses ALY-GFP

The present inventors have further discovered the cellular factor ALY is reversibly transported across the nuclear membrane such that when ALY binds to mRNA, the resultant complex is exported from the nucleus, the ALY is released and ALY returns to the nucleus where it can combine with another mRNA for export. In certain preferred embodiments of the invention, ALY co-localizes with essential splicing factor SC35 in nuclear speckle domains as shown by double indirect immunofluorescence microscopy.

In a preferred embodiment of the present invention, the nuclear export of nonspliced mRNA in a metazoan cell is increased by providing an excess quantity of recombinant cellular factor ALY in the cell. The excess recombinant cellular factor ALY promotes the formation of an ALY-mRNA binding complex and facilitates export of the mRNA from the nucleus to the cytoplasm of the metazoan cell. The ALY-mRNA binding complex is more readily exported from the nucleus than mRNA not in contact with recombinant cellular factor ALY thereby facilitating mRNA export relative to a metazoan cell devoid of excess recombinant cellular factor ALY. After export of mRNA as part of the ALY-mRNA binding complex the recombinant cellular factor ALY can subsequently dissociate from the binding complex and relocalize into the nucleus.

Any recombinant protein can be expressed in accord with the present invention. Thus, the metazoan cell can be transfected with the cDNA of any gene for which expression is desired. Non-limiting examples of cDNA or mRNA sequences that can be used in accordance with the invention include those that encode, express or transcribe any commercial or therapeutic desirable protein. Examples of such proteins include: Factor VIII, Factor IX, Interleukin-11, erythropoetin, Gm-CSF, BMP (a bone morphogenic protein).

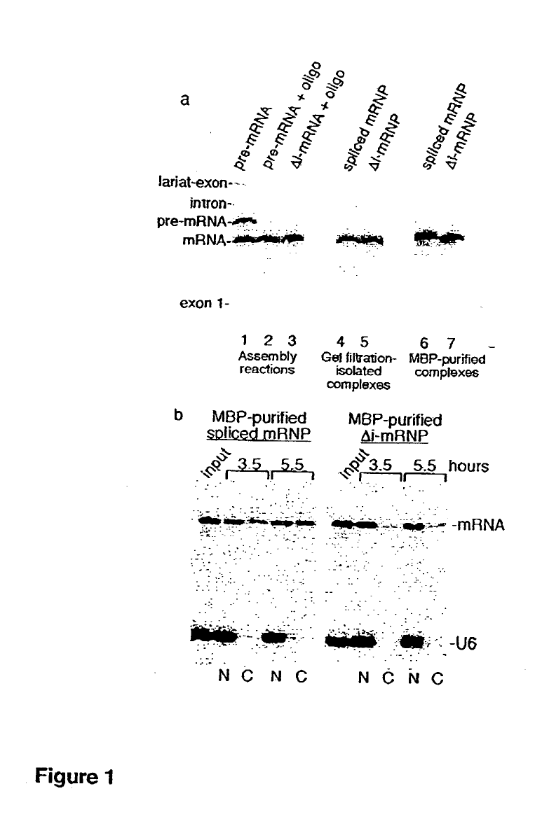

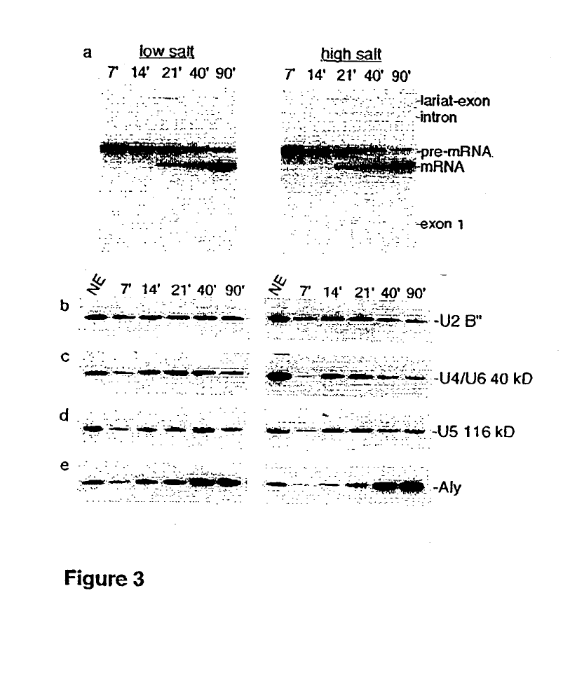

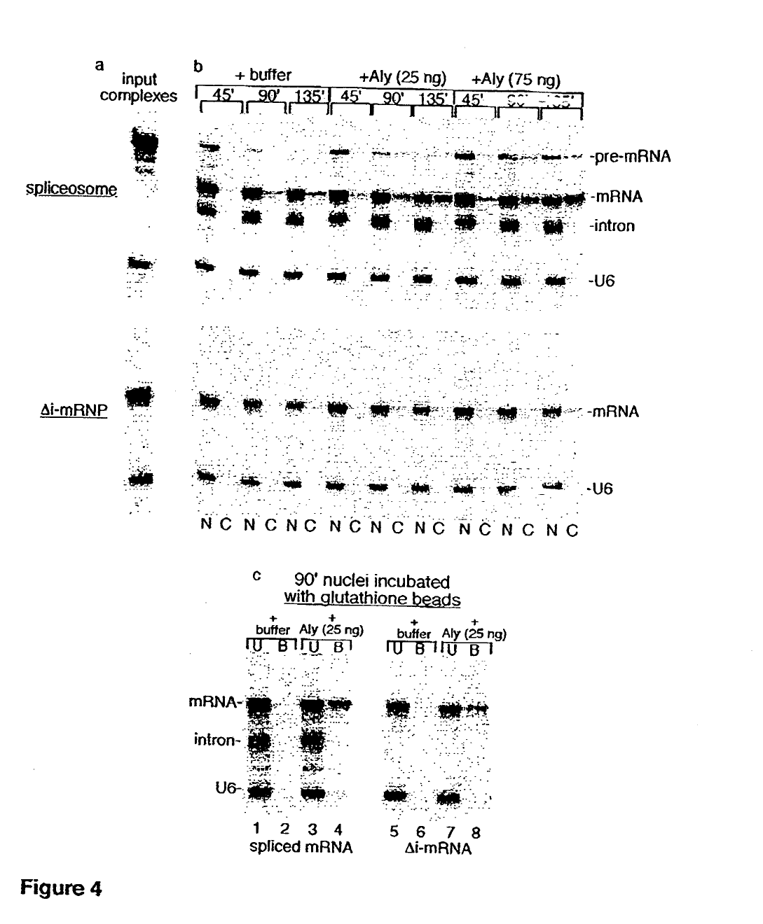

Determination of the mRNA content of the nucleus and cytoplasm of a metazoan cell can be determined by any method that permits distinction between the nuclear mRNA and the cytoplasmic mRNA. Preferable methods of analysis for mRNA content in the nucleus and cytoplasm are fractionation of mRNA on 8% (FIG. 1b and FIG. 4) or 15% (FIG. 1a and FIG. 3a) denaturing polyacrylamide gels or binding of mRNA contained in a (GST-ALY)-mRNA binding complex to glutathione beads.

In the export of spliced mRNA facilitated by the recombinant cellular factor ALY, the contacting of ALY to mRNA to form the ALY-mRNA binding complex preferably is promoted by the splicing machinery during intron excision of a spliceosome. In accord with the present invention, recombinant cellular factor ALY has high specificity for facilitating the export of mRNA in presence of other RNA sequences including but not limited to snRNA, tRNA, pre-spliced mRNA, and the spliced intron sequence. Addition of excess recombinant ALY to the metazoan cell generally does not reduce the specificity of ALY for the selective export of mRNAs. For example, in the presence of U6 snRNA, pre-spliced mRNA and spliced intron within the nucleus of a metazoan cell containing 25 ng of recombinant cellular factor ALY (25 ng) only spliced mRNA is exported from the nucleus leaving the U6 snRNA, pre-spliced mRNA and spliced intron in the nucleus of the cell. When the metazoan cell comprises a higher quantity of recombinant cellular factor ALY (e.g., 75 ng), a low level of pre-spliced mRNA was exported in addition to spliced mRNA. Regardless of the quantity of recombinant cellular factor ALY present in the metazoan cell, tRNA and U6 snRNA were not exported from the nucleus.

Remarkably, the present invention discovered that addition of excess recombinant ALY increases rate and efficiency of spliced mRNA export from the nucleus of a metazoan cell (see Example 4). This stimulation of export was not observed with any other proteins including but not limited to hnRNP A1 or GST-Dbp5. The addition of excess recombinant cellular factor ALY to a metazoan cell increases the promotion of spliced mRNA export in a dose-dependant manner.

Thus, the present invention provides a method for increasing the level of gene expression obtained in a metazoan cell. Providing a recombinant cellular factor ALY in the cell allows the transcribed mRNA sequence from the expressed gene to be exported more rapidly and/or more efficiently from the nucleus. The level of transcribed mRNA exported into the cytoplasm of a cell containing recombinant ALY thereby increases the number of templates for protein transcription and, thus, the ultimate level of gene expression.

Thus, the present invention provides a method for obtaining higher levels of recombinant protein from a DNA sequence encoding said protein in a metazoan cell by providing recombinant cellular factor ALY in the cell producing the protein. The recombinant cellular factor ALY complexes with mRNA transcribed from the DNA sequence that facilitates export of the bound mRNA sequence relative to uncomplexed mRNA sequences thus providing higher levels of protein transcription. Purification of the proteins is accomplished by standard purification techniques that are well known within the art.

The present invention is further illustrated by the following examples. These examples are provided to aid in the understanding of the invention and are not to be construed as limitations thereof.

### EXAMPLE 1

### Isolation of Substantially Purified ALY-mRNA Binding Complex

Pre-mRNAs were synthesized in vitro using ³²P-labeled UTP and T7 RNA polymerase under standard conditions. Adenovirus Major Late (AdML) pre-mRNA was incubated in nuclear extracts under splicing conditions for 60′ (FIG. 1, lane 1). AdML pre-mRNA was incubated under splicing conditions for 60′, followed by addition of a 12-mer oligonucleotide (5′ CTGCCTTTAGTG 3′) complementary to intron sequences immediately downstream from the 5′ splice site (FIG. 1, lanes 2 and 3). Incubation was continued for 30′ which results in RNase H digestion of the pre-mRNA, intron lariat, and lariat-intermediate (compare lanes 1 and 2 in FIG. 1). The complex shown in lane 2 was then isolated by gel filtration on Sephacryl S-500 (Reed, PNAS (1990) 87:8021-5). Gel filtration-isolated mRNPs were further purified by maltose binding protein (MBP) affinity chromatography. The mRNPs comprise ALY and AdML spliced mRNA whereby the composition was confirmed by increased rate of export from the nucleus of Xenopus and Western analysis. A mixture of the purified spliced AdML mRNP and U6 snRNA, which is retained in the nucleus (Hamm and Mattaj, Cell v. 63, p.109-18, 1990) was injected into Xenopus oocyte nuclei (FIG. 1b, lanes input). The mRNA in the spliced mRNP is rapidly and efficiently exported while the U6 snRNA is not exported. Western analysis using the cellular factor ALY antibody (Bruhn, GENES DEV (1997) 11:640-53) was performed and cellular factor ALY was detected in the mRNP generated by splicing of AdML pre-mRNA.

### EXAMPLE 2

### Determination of ALY Binding Selectivity for mRNA in the Presence of Other RNA Sequences

AdML pre-mRNA was incubated under splicing conditions in vitro for 20 minutes to assemble the spliceosome. Aliquots of the reactions were mixed with U6 snRNA, and total RNA was analyzed. GST-ALY (25 ng) or buffer (control) alone was injected into the Xenopus oocyte cytoplasm followed by overnight incubation to import ALY into the nucleus. AdML pre-mRNA complex was injected into the nuclei of these oocyctes followed by incubation for 90′. The nuclei were isolated, homogenized in binding buffer (20 mM HEPES-NaOH, pH 7.9, 60 mM KCl, 0.1% Triton X-100) and rotated with glutathione beads overnight. Total RNA in bound and unbound fractions was analyzed. In the nuclei that contained GST-ALY, only spliced mRNA was bound to the glutathione beads with no detectable binding of the intron, tRNA or U6 snRNA.

### EXAMPLE 3

An experiment was conducted using conditions similar to Example 2 wherein nonspliced mRNA was substituted for AdML pre-mRNA. In the nuclei that comprised GST-ALY, nonspliced mRNA was bound to the glutathione beads with no detectable binding of tRNA or U6 snRNA.

### EXAMPLE 4

### Increased Rate of mRNA Export in the Presence of Excess Recombinant Cellular Factor ALY

AdML pre-mRNA was incubated under splicing conditions in vitro for 20 minutes to assemble the spliceosome. Aliquots of the reaction were mixed with U6 snRNA, and total RNA was analyzed. GST-ALY (25 or 75 ng) or buffer alone was injected into the Xenopus oocyte cytoplasm followed by overnight incubation to import ALY. The RNA mixture was injected into the nuclei of these oocyctes followed by incubation for 45′, 90′ and 135′. RNA isolated from the nucleus (N) and cytoplasm (C) were analyzed separately. GST-ALY increases both the rate and efficiency of spliced mRNA export (FIGS. 4b, and c). The export of tRNA is not affected by excess GST-ALY, indicating that ALY is specific for mRNA export.

### EXAMPLE 5

Nonspliced mRNA was incubated under splicing conditions in vitro for 20 minutes to assemble nonspliced mRNP. Aliquots of the reaction were mixed with U6 snRNA, and total RNA was analyzed. GST-ALY (25 or 75 ng) or buffer alone was injected into the Xenopus oocyte cytoplasm followed by overnight incubation to import ALY. The RNA mixture was injected into the nuclei of these oocyctes followed by incubation for 45 minutes, 90 minutes and 135 minutes. RNA isolated from the nucleus (N) and cytoplasm (C) were analyzed separately. Nonspliced mRNA is not exported efficiently (FIGS. 4b and c). Interestingly, slightly more nonspliced mRNA export is observed in the presence of excess ALY (FIGS. 4b and c). This stimulation occurs because GST-ALY is present in excess.

### EXAMPLE 6

### ALY Shuttles Between the Nucleus and the Cytoplasm

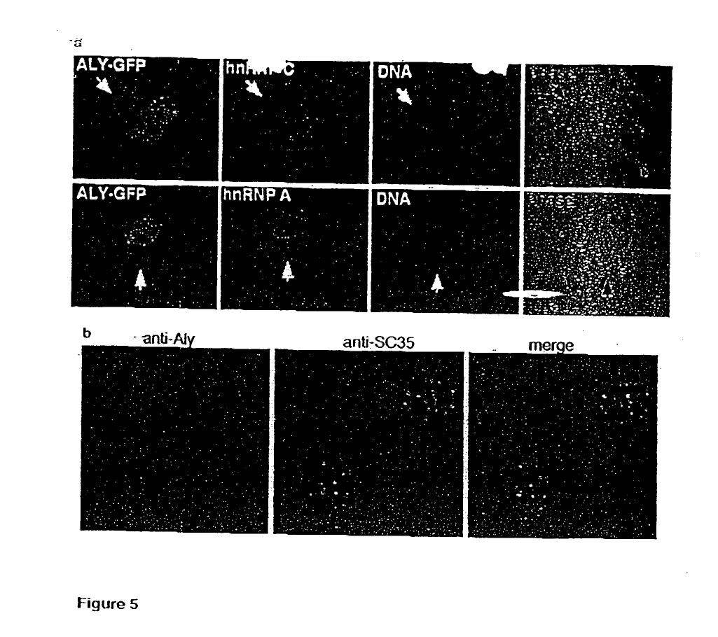

To determine whether ALY shuttles between the nucleus and cytoplasm, we carried out a heterokaryon shuttling assay wherein a murine fibrosarcoma cell line L929 stably expressing ALY-GFP was co-cultured with Xenopus A6 cells in the presence of 100 mg/ml cycloheximide. Cells were fused by PEG 1500 and incubated in the presence of cycloheximide for 4 hrs (Katahira, EMBO J (1999) 18:1593-606) and Pinol-Roma, Nature (1992) 355:730-2). The cells were fixed, permeabilized, and incubated with hnRNP C monoclonal antibodies followed by incubation with cy3-labeled anti-mouse IgG. ALY was detected in the Xenopus and murine nuclei in the heterokaryon whereas hnRNP C remains in the murine nucleus (FIG. 5a). Thus, it can be concluded that ALY shuttles.

### EXAMPLE 7

An experiment was carried out using conditions similar to Example 6 wherein hnRNP C monoclonal antibodies were substituted with hnRNP A1. Both ALY and hnRNA A1 were detected in the Xenopus and murine nuclei in the heterokaryon (FIG. 5a). Thus, it can be concluded that ALY shuttles.

### EXAMPLE 8

### Determination of ALY Nuclear Distribution

As shown by double indirect immunofluorescence of HeLa cells using affinity-purified ALY or SC35 antibodies, ALY is present in nuclear speckles and co-localizes with the essential splicing factor SC35 (FIG. 5b) (Fu and Maniatis, Nature (1990) 343:437-41). The immunofluorescence microscopy was carried out as previously described for other systems hrs (Katahira, EMBO J (1999) 18:1593-606) and as is well known to those skilled in the art.

### EXAMPLE 9

### Determining the Timing of ALY Recruitment to mRNA During Splicng

AdML pre-mRNA was incubated under splicing conditions for the indicated times. The complexes at each time point were isolated by gel filtration followed by MBP-affinity purification in low (60 mM) or high (250 mM) salt concentration. a. RNA from the eluted complexes was analyzed. b-e. Western blots of total protein from the eluted complexes shown in a were probed with the indicated antibodies. Only unspliced pre-mRNA is detected in the 7′ and 14′ spliceosomes. By 21′, splicing intermediates and low levels of spliced mRNA are detected. Finally, spliced mRNA accumulates by 40′ and is the main RNA species at 90′ Western analysis of the purified complexes using antibodies to the U2 snRNP-specific B″ protein and U5 snRNP-specific 40 K and 116 K proteins (Habets, J Immunol (1989) 143:2560-6; Achsel, Mol Cell Biol (1998) 18:6756-6; Fabrizio, EMBO J (1997) 16:4092-106). U2 snRNP is tightly associated with the spliceosome at 7′ whereas a lower level of U5 snRNP is present (FIGS. 3b,c and d). At later time points, both snRNPs are present. The level of snRNP present is similar at high and low salt concentration. In contrast to the snRNP proteins, the levels of ALY are significantly different throughout the time course and in low and high salt (FIG. 3e). In low salt, ALY is first detected in the 7′ spliceosome and is more abundant at the later time points. In high salt, ALY is tightly associated only with the complexes containing high levels of spliced mRNA (40′ and 90′ complexes). ALY is recruited during spliceosome assembly and then becomes more tightly associated with the spliced mRNP.

The invention has been described in detail with reference to the preferred embodiments. However, it will be realized that upon consideration of the specification and figures, those skilled in the art may make modifications and improvements within the spirit and scope of this invention. Thus, it should be realized that the present invention is not limited to any particular source of a recombinant cellular factor ALY or to any particular method for the introduction of recombinant cellular factor ALY into a metazoan cell.

The disclosures of the publications listed below are hereby incorporated by reference.

1. Luo, M. & Reed, R. Splicing is required for rapid and efficient mRNA export in metazoans. Proc Natl Acad Sci U S A 96, 14937-14942 (1999).

2. Strasser, K. & Hurt, E. Yralp, a conserved nuclear RNA-binding protein, interacts directly with Mex67p and is required for mRNA export. EMBO J 19, 410420 (2000).

3. Le Hir, H., Moore, M. J. & Maquat, L. E. Pre-mRNA splicing alters mRNP composition: evidence for stable association of proteins at exon-exon junctions. Genes Dev 14, 1098-1108 (2000).

4. Das, R., Zhou, Z. & Reed, R. Functional association of U2 snRNP with the ATP-independent spliceosomal complex E. Molecular Cell 5, 779-787 (2000).

5. Hamm, J. & Mattaj, I. W. Monomethylated cap structures facilitate RNA export from the nucleus. Cell 63, 109-118 (1990).

6. Stutz, F. et al. REF, an evolutionary conserved family of hnRNP-like proteins, interacts with TAP/Mex67p and participates in mRNA nuclear export. RNA 6, 638-650 (2000).

7. Segref, A. et al. Mex67p, a novel factor for nuclear mRNA export, binds to both poly(A)+RNA and nuclear pores. EMBO J 16, 3256-3271 (1997).

8. Gruter, P. et al. TAP, the human homolog of Mex67p, mediates CTE-dependent RNA export from the nucleus. Mol Cell 1, 649-659 (1998).

9. Ernst, R. K., Bray, M., Rekosh, D. & Hammarskjold, M. L. A structured retroviral RNA element that mediates nucleocytoplasmic export of intron-containing RNA. Mol Cell Biol 17, 135-144 (1997).

10. Pasquinelli, A. E. et al. The constitutive transport element (CTE) of Mason-Pfizer monkey virus (MPMV) accesses a cellular mRNA export pathway. EMBO J 16, 7500-7510 (1997).

11. Saavedra, C., Felber, B. & Izaurralde, E. The simian retrovirus-1 constitutive transport element, unlike the HIV-1 RRE, uses factors required for cellular mRNA export. Curr Biol 7, 619-628 (1997).

12. Bruhn, L., Munnerlyn, A. & Grosschedl, R. ALY, a context-dependent coactivator of LEF-1 and AML-1, is required for TCRalpha enhancer function. Genes Dev 11, 640-653 (1997).

13. Wichmann, I., Garcia-Lozano, J. R., Respaldiza, N., Gonzalez-Escribano, M. F. & Nunez-Roldan, A. Autoantibodies to transcriptional regulation proteins DEK and ALY in a patient with systemic lupus erythematosus. Hum Immunol 60, 57-62 (1999).

14. Portman, D. S., O'Connor, J. P. & Dreyfuss, G. YRA1, an essential Saccharomyces cerevisiae gene, encodes a novel nuclear protein with RNA annealing activity. RNA 3, 527-537 (1997).

15. Fu, X. D. The superfamily of arginine/serine-rich splicing factors. Rna 1, 663-680 (1995).

16. Bennett, M., Pinol-Roma, S., Staknis, D., Dreyfuss, G. & Reed, R. Differential binding of heterogeneous nuclear ribonucleoproteins to mRNA precursors prior to spliceosome assembly in vitro. Mol Cell Biol 12, 3165-3175 (1992).

17. Dreyfuss, G., Matunis, M. J., Pinol-Roma, S. & Burd, C. G. hnRNP proteins and the biogenesis of mRNA. Annu Rev Biochem 1993;62:289-321 (1993).

18. Habets, W. J., Hoet, M. H., De Jong, B. A., Van der Kemp, A. & Van Venrooij, W. J. Mapping of B cell epitopes on small nuclear ribonucleoproteins that react with human autoantibodies as well as with experimentally-induced mouse monoclonal antibodies. J Immunol 143, 2560-2566 (1989).

19. Achsel, T., Ahrens, K., Brahms, H., Teigelkamp, S. & Luhrmann, R. The human U5-220kD protein (hPrp8) forms a stable RNA-free complex with several U5-specific proteins, including an RNA unwindase, a homologue of ribosomal elongation factor EF-2, and a novel WD-40 protein. Mol Cell Biol 18, 6756-6766 (1998).

20. Fabrizio, P., Laggerbauer, B., Lauber, J., Lane, W. S. & Luhrmann, R. An evolutionarily conserved U5 snRNP-specific protein is a GTP-binding factor closely related to the ribosomal translocase EF-2. EMBO J 16, 4092-4106 (1997).

21. Staley, J. P. & Guthrie, C. Mechanical devices of the spliceosome: motors, clocks, springs, and things. Cell 92, 315-326 (1998).

22. Kang, Y. & Cullen, B. R. The human Tap protein is a nuclear mRNA export factor that contains novel RNA-binding and nucleocytoplasmic transport sequences. Genes Dev 13, 1126-1139 (1999).

23. Katahira, J. et al. The Mex67p-mediated nuclear mRNA export pathway is conserved from yeast to human. EMBO J 18, 2593-2609 (1999).

24. Spector, D. L. Macromolecular domains within the cell nucleus. Annu Rev Cell Biol 9, 265-315 (1993).

25. Fu, X. D. & Maniatis, T. Factor required for mammalian spliceosome assembly is localized to discrete regions in the nucleus. Nature 343, 437-441 (1990).

26. Lewis, J. D. & Tollervey, D. Like attracts like: getting RNA processing together in the nucleus. Science 288, 1385-1389 (2000).

27. Nakielny, S. & Dreyfuss, G. Transport of proteins and RNAs in and out of the nucleus. Cell 99, 677-690 (1999).

28. Pinol-Roma, S., Choi, Y. D., Matunis, M. J. & Dreyfuss, G. Immunopurification of heterogeneous nuclear ribonucleoprotein particles reveals an assortment of RNA-binding proteins [published erratum appears in Genes Dev 1988 Apr;2(4):490]. Genes Dev 2, 215-227 (1988).

29. Pinol-Roma, S. & Dreyfuss, G. Shuttling of pre-mRNA binding proteins between nucleus and cytoplasm. Nature 355, 730-732 (1992).

30. Reed, R. Protein composition of mammalian spliceosomes assembled in vitro. Proc Natl Acad Sci U S A 87, 8031-8035 (1990).

What is claimed is:

1. A therapeutic composition comprising in a pharmaceutically acceptable carrier a complex comprising recombinant cellular factor ALY and mRNA.

2. A therapeutic composition of claim 1, wherein the mRNA is transcribed from DNA comprising an intron.

3. A therapeutic composition of claim 1, wherein the composition is non-pyrogenic

4. A metazoan cell comprising a recombinant cellular factor ALY.

5. A metazoan cell of claim 4, wherein the metazoan cell is a mammalian cell.

6. A metazoan cell of claim 4, wherein at least a portion of the recombinant cellular factor ALY binds to mRNA to form a complex comprising recombinant cellular factor ALY and mRNA.

7. A metazoan cell of claim 4, wherein the cell further comprises a complex of recombinant cellular factor ALY and mRNA where the mRNA had an intron spliced therefrom.

8. The metazoan cell of claim 4, wherein at least a portion of the recombinant cellular factor ALY added to the metazoan cell localizes to the nucleus.

9. The metazoan cell of claim 4, further comprising a vector that expresses ALY.

10. The metazoan cell of claim 9, wherein the vector is localized in the nucleus.

11. The metazoan cell of claim 9, wherein the vector is localized in the cytoplasm.

12. The metazoan cell of claim 4, wherein the metazoan cell comprises at least about 5 ng of the recombinant cell factor ALY.

13. The metazoan cell of claim 4, wherein the metazoan cell comprises at least about 25 ng of the recombinant cell factor ALY.

14. The metazoan cell of claim 4, wherein the metazoan cell comprises at least about 75 ng of the recombinant cell factor ALY.

15. A metazoan cell comprising (a) recombinant DNA that expresses a preselected protein and (b) recombinant cellular factor ALY.

16. A metazoan cell of claim 15, wherein the metazoan cell is a mammalian cell.

17. A metazoan cell of claim 15, further comprising a vector that expresses cellular factor ALY.

18. A method for increasing the export of mRNA from the nucleus of a metazoan cell, the method comprising introducing recombinant cellular factor ALY into a metazoan cell.

19. A method of claim 18, wherein the metazoan cell is a mammalian cell.

20. A method of claim 18, wherein at least a portion of the recombinant cellular factor ALY binds to mRNA to facilitate nuclear export.

21. A method of claim 18, wherein binding the recombinant cellular factor ALY to mRNA is mediated by splicing an intron from a pre-mRNA sequence.

22. A method of claim 18, wherein recombinant cellular factor ALY is introduced into either the cytoplasm or nucleus of the metazoan cell.

23. A method of claim 18, wherein recombinant cellular factor ALY is introduced into the nucleus of the metazoan cell.

24. The method of claim 18, wherein the recombinant cellular factor ALY is introduced into the metazoan cell by microinjection of a vector that expresses ALY.

25. The method of claim 24, wherein vector is microinjected into the cytoplasm of the metazoan cell.

26. The method of claim 25, wherein at least a portion of the recombinant cellular factor ALY expressed into the cytoplasm of the metazoan cell is transported into the nucleus.

27. The method of claim 24, wherein the vector is microinjected into the nucleus of the metazoan cell.

28. The method of claim 24, wherein ALY shuttles between the nucleus and the cytoplasm.

29. The method of claim 18, wherein recombinant cellular factor ALY is introduced into the metazoan cell by fusing the metazoan cell with a cell that stably expresses the recombinant cellular factor ALY.

30. A method for increasing gene expression in a metazoan cell, the method comprises the step of introducing recombinant cellular factor ALY into a metazoan cell wherein at least a portion of the recombinant cellular factor ALY is transported into in the nucleus, binds to mRNA to form a complex wherein the recombinant cellular factor ALY-mRNA complex formation facilitates export of mRNA from the nucleus thereby increasing gene expression.

31. A method for obtaining higher levels of recombinant proteins from a metazoan cell, the method comprises the step of introducing recombinant cellular factor ALY into a metazoan cell wherein at least a portion of the recombinant cellular factor ALY is transported into in the nucleus, binds to mRNA to form a complex wherein the recombinant cellular factor ALY-mRNA complex formation facilitates export of mRNA from the nucleus thereby increasing recombinant protein expression, and purifying recombinant protein produced from increased export of mRNA.
